(19) United States
(12) Patent Application Publication
(10) Pub. No.: US 2016/0051462 A1
(43) Pub. Date: Feb. 25, 2016

(54) COSMETIC OR DERMATOLOGICAL USE OF A POLYGONUM BISTORTA EXTRACT

(71) Applicants: BASF BEAUTY CARE SOLUTIONS FRANCE SAS, Lyon (FR); UNIVERSITE CLAUDE BERNARD LYON I, Villeurbanne Cedex (FR); CENTRE NATIONAL DE LA RECHERCHE SCIENTIFIQUE, Paris (FR)

(72) Inventors: Valérie ANDRE-FREI, Ampuis (FR); Nicolas BECHETOILLE, Brignais (FR); Sabine PAIN, Vienne (FR); Patricia ROUSSELLE, Lyon (FR)

(21) Appl. No.: 14/780,251

(22) PCT Filed: Mar. 27, 2014
(86) PCT No.: PCT/FR2014/050731

§ 371 Date: Sep. 25, 2015

(30) Foreign Application Priority Data
Mar. 27, 2013 (FR) 1352769

(51) Int. Cl. A61K 8/97; A61Q 19/00; A61Q 19/08; A61K 36/704; A61K 8/34

(52) U.S. Cl. CPC A61K 8/97; A61K 36/704; A61K 8/345; A61Q 19/08; A61Q 19/00; A61K 2800/805; A61K 2800/74; A61K 2800/596

(57) ABSTRACT

The present invention relates to the topical cosmetic use of a Polygonum bistorta extract for stimulating the expression of perlecan and/or dystroglycan, in particular in the extracellular matrix and/or in the epithelial basement membrane, especially the dermoepidermal junction.

It also relates to a cosmetic care method comprising the application, to at least one concerned area of healthy skin and/or healthy mucous membrane and/or healthy scalp, of a Polygonum bistorta extract or of a cosmetic composition comprising such an extract for stimulating the expression of perlecan and of dystroglycan, in particular in the extracellular matrix and/or in the epithelial basement membrane, especially the dermoepidermal junction.

It finally relates to a Polygonum bistorta extract or a dermatological composition containing a Polygonum bistorta extract and a dermatologically acceptable excipient for topical use in the treatment and/or prevention of rosacea, telangiectasias, chapping and/or disorders of the buccal and/or ocular mucous membranes.

The invention relates to the topical cosmetic or dermatological use of a Polygonum bistorta extract, for stimulating the expression of perlecan and/or dystroglycan, in particular in the extracellular matrix and/or in the epithelial basement membrane, especially the dermoepidermal junction.

Among the heparan sulfate proteoglycans (HSPGs) of basal laminae, perlecan has a major role in morphogenesis of the epithelium, in particular the epidermis, but also in the survival, proliferation and differentiation of keratinocytes and of endothelial cells, which are in particular cutaneous. Perlecan regulates these processes by controlling the bioavailability of growth factors.

Dystroglycan is a glycoprotein that is a potential receptor for perlecan. Perlecan and dystroglycan are expressed on basement membranes, which are ubiquitous structures located in various tissues, such as epithelia and endothelia, but also in various cell types such as keratinocytes, fibroblasts and endothelial cells. They interact together and contribute to ensuring the solidity and stability of the structure of the skin, of the mucous membranes and of the scalp, in particular the epithelium, and especially the epidermis and the dermis. As it happens, during ageing, in particular chronobiological ageing, the expression of perlecan and dystroglycan is drastically reduced. In particular, the expression of perlecan decreases greatly at the level of the dermoepidermal junction and the dermal capillaries with age. This decrease is not due to degradation of the protein, but to a decrease of its expression, and quite particularly in its transcriptional regulation, by keratinocytes and endothelial cells. Moreover, the inventors have noted that there is a correlation between the lack of synthesis of perlecan and the thickness of the skin and/or of the mucous membranes and/or of the scalp, in particular of the epithelium, preferentially the epidermis. The expression of perlecan and dystroglycan is therefore of great importance in skin and/or mucous membrane and/or scalp homeostasis and in maintaining their firmness and their density, which are degraded during ageing, in particular chronobiological ageing.

Surprisingly, the inventors have discovered that a Polygonum bistorta extract stimulates the expression of HSPGs and glycoproteins of basal laminae, and more particularly of perlecan and of dystroglycan, in particular in the extracellular matrix and/or in the epithelial basement membrane, especially the dermoepidermal junction, more particularly in skin and/or mucous membrane and/or scalp keratinocytes and/or endothelial cells, which are preferentially cutaneous. Targeting both keratinocytes and endothelial cells, which are in particular cutaneous, makes it possible to have a double effect: 1) on improving the microvascular pathway for nourishing the skin, the mucous membranes and/or the scalp and 2) on improving the complexion in terms in particular of luminosity. Targeting not only perlecan but also dystroglycan makes it possible, in addition, to act completely on the pathway of expression and activity of perlecan.

Polygonum bistorta is a plant which is found in Europe except in the Mediterranean region, in Asia and in North America up to an altitude of 2400 m. It grows in wet and mountainous pastures, at the edge of streams, on nitrogen-rich soils. It is known for its analgesic effects when administered peritoneally, as a sedative and astringent, and is described as a powerful, healing and anti-diarrhea tonic.

Application FR2867977 describes cosmetic, pharmaceutical and dermocosmetic compositions intended for preventing and/or combating the formation of fine lines and wrinkles on the skin by limiting contractions of the subcutaneous muscles, containing resveratrol and/or certain derivatives thereof. Among the numerous plant extracts that may contain resveratrol and/or these derivatives, mention is made of Polygonum bistorta. Nevertheless, the effect described in said patent is a mechanical effect due to a muscle-relaxing action as an alternative to the use of botulinum toxin. It therefore involves a mechanism of action and a result that are very different than the route of action on the expression of perlecan and of dystroglycan according to the invention which results in an increase in firmness and in density, effects that are opposite to the slackening of the skin observed with a myoinhibitor. In addition, it should be noted that dystroglycan also contributes to muscle contraction; thus, increasing its expression therefore induces an opposite effect to myoinhibition.

The present invention therefore relates to the topical cosmetic or dermatological use of a Polygonum bistorta extract for stimulating the expression of perlecan and/or dystroglycan, in particular in the extracellular matrix and/or in the epithelial basement membrane, especially the dermoepidermal junction.

Preferentially, the use according to the invention is cosmetic and by topical application to at least one concerned area of healthy skin and/or of a healthy mucous membrane and/or of healthy scalp, in particular of a human being. Preferentially, the Polygonum bistorta extract is obtained from the root, preferentially obtained by aqueous extraction.

Preferentially, the use of Polygonum bistorta stimulates the expression of perlecan and/or dystroglycan in keratinocytes and/or endothelial cells of the skin and/or of mucous membranes and/or of the scalp, preferably endothelial cells of the skin.

The plant extract according to the invention can be extracted from the whole of the plant or from one or more parts of the plant, in particular chosen from the root, the stem, the bark, the flower, the seed, the germ and/or the leaf and mixtures thereof. The plant extract according to the invention is preferentially extracted from the roots.

The extract may therefore be obtained by plant extraction methods known in the field, for example by maceration of at least one part of the plant preferably between 1% and 10% (p/p) in a solvent or a mixture of solvents, preferably a protic polar solvent, and advantageously in water, an alcohol, a glycol, a polyol, or a water/alcohol, water/glycol or water/polyol mixture (such as water as a mixture with ethanol, glycerol, butylene glycol or other glycols, such as xylitol, etc.) of 100/0 to 0/100 (v/v). The extract is preferentially obtained by aqueous extraction.

For the purposes of the present invention, the term “Polygonum bistorta extract obtained by aqueous extraction” is intended to mean any extract obtained by extraction with an aqueous solution, in particular by maceration in an aqueous solution, containing more than 60% by weight, advantageously at least 70% by weight, in particular at least 80% by weight, more particularly at least 90% by weight, particularly at least 95% by weight, of water relative to the total weight of the aqueous solution, even more advantageously not containing butylene glycol, in particular not containing alcohol, more particularly containing only water.

According to one advantageous embodiment, the extract according to the invention is obtained by cold maceration, preferentially at 4° C., or at ambient temperature, i.e. between 18 and 25° C., preferentially 20° C., optionally after a step of drying the plant. According to one preferred mode, the extract according to the invention is obtained by maceration at ambient temperature, preferentially 20° C.

According to one preferential embodiment, the extract according to the invention is obtained by maceration for a period of between 30 minutes and 24 hours, preferentially between 1 hour and 5 hours, more preferentially 2 hours.

The extract obtained is then preferably centrifuged and/or filtered and/or distilled in order to recover the active soluble fraction. It is preferentially filtered at a cut-off threshold of 0.45 μm, more preferentially 0.22 μm. Additional discoloring and/or deodorizing steps can be carried out on the extract at any stage of the extraction and according to techniques known to those skilled in the art.

The extract according to the invention may also be subsequently concentrated by evaporation of the solvent, for example by lyophilization or by atomization.

Particularly advantageously, the amount of plant during the extraction, preferably the root, is 1% by weight relative to the total weight of the mixture of plant/extraction solvent, preferentially aqueous solution. In particular, the plant is milled before extraction. Advantageously, the extraction lasts 2 hours and is carried out at ambient temperature, particularly 20° C. Even more advantageously, the extract according to the invention contains only traces of resveratrol and/or trans-stilbene (C₁₄H₁₂, MW 180.24 g/mol) and/or resveratrol derivatives in particular in the form of resveratrol oligomers and/or resveratrol analogs, such as rhapontin (of formula C₂₁H₂₄O₉, MW 420.14 g/mol), deoxyrhapontin (C₂₁H₂₄O₈, MW 404.14 g/mol), epsilon-viniferin, resveratrol acetates, formic esters and glucosides, or 3,4′,5-trihydroxystilbene-3-O-beta-D-glucopyranoside (C₂₀H₂₂O₈, MW 390.13 g/mol), in an insufficient amount to have an effect on muscle contraction, in particular in an amount of less than 0.01% by weight, preferentially less than 0.001% by weight of the extract according to the invention. Preferentially, the extract according to the invention does not contain resveratrol and/or trans-stilbene (C₁₄H₁₂, MW 180.24 g/mol) and/or resveratrol derivatives in particular in the form of resveratrol oligomers and/or resveratrol analogs, such as rhapontin (of formula C₂₁H₂₄O₉, MW 420.14 g/mol), deoxyrhapontin (C₂₁H₂₄O₈, MW 404.14 g/mol), epsilon-viniferin, resveratrol acetates and glucosides, and 3,4′,5-trihydroxystilbene-3-O-beta-D-glucopyranoside (C₂₀H₂₂O₈, MW 390.13 g/mol).

The extract obtained is preferentially soluble in a solvent, in particular a polar solvent, such as water, an alcohol, a polyol, a glycol, or a mixture thereof, preferentially an aqueous-glycolic mixture, more preferentially containing a glycol chosen from caprylyl glycol, hexylene glycol and mixtures thereof. Advantageously, the extract is soluble in an aqueous solution containing hexylene glycol, in particular containing between 0.1% and 10% by weight of hexylene glycol relative to the total weight of the aqueous solution, more particularly between 1% and 5% by weight of hexylene glycol relative to the total weight of the aqueous solution. Advantageously, the extract is soluble in an aqueous solution containing caprylyl glycol, in particular containing between 0.01% and 5% by weight of caprylyl glycol relative to the total weight of the aqueous solution, more particularly between 0.1% and 1% by weight of caprylyl glycol relative to the total weight of the aqueous solution. Advantageously, the aqueous solution does not contain butylene glycol.

For the purposes of the present invention, the term “healthy skin”, “healthy mucous membrane” or “healthy scalp” is intended to mean an area of skin, mucous membrane or scalp to which the extract according to the invention is applied and which is termed “non-pathological” by a dermatologist, i.e. not exhibiting any skin infection, disease or condition such as candidiasis, impetigo, psoriasis, eczema, acne or dermatitis, or any wounds or injuries. Preferentially, for the purposes of the present invention, the term “cosmetic” is intended to mean non-therapeutic use, i.e. use on healthy skin, healthy mucous membrane and/or a healthy scalp.

For the purposes of the present invention, the term “topical” is intended to mean the application of the Polygonum bistorta extract and/or of the cosmetic composition containing such an extract to the surface of the concerned area of the skin and/or mucous membranes and/or scalp, in particular of a human being, in particular by direct application or by spraying. For the purposes of the present invention, the expression “stimulation of the expression of perlecan and/or of dystroglycan” is intended to mean an increase in the gene and/or protein expression respectively of perlecan and/or of dystroglycan, preferentially in the protein expression. This increase can be measured on a model, comprising at least one cell type exhibiting expression of perlecan and/or of dystroglycan, advantageously keratinocytes and/or endothelial cells, which are preferentially cutaneous, in contact with the Polygonum bistorta extract according to the invention, and is reflected by an increase in the respective gene and/or protein expression of perlecan and/or of dystroglycan greater than or equal to 10%, advantageously greater than or equal to 20%, relative to the level of gene and/or protein expression in a control model, i.e. without bringing into contact with the Polygonum bistorta extract according to the invention. The increase in this expression is preferentially a protein expression increase. Such models are described in the examples.

According to the invention, the term “mucous membranes” denotes especially the buccal mucous membrane, especially the buccal, labial, nasal, ocular, anal and/or urogenital mucous membranes, in particular the labial mucous membrane.

Advantageously, the use according to the present invention is for preventing and/or combating ageing of the skin and/or of the mucous membranes and/or of the scalp, in particular chronobiological and/or photobiological ageing, for preventing and/or combating a decrease in homeostasis of the skin and/or of the mucous membranes and/or of the scalp and/or for improving it, in particular in the epidermis, for reinforcing the epithelial basement membrane of the skin and/or of the mucous membranes and/or of the scalp, preferentially the dermoepidermal junction, for improving keratinocyte proliferation and/or differentiation, especially at the epidermal level, in particular associated with ageing of the skin and/or of the mucous membranes and/or of the scalp, for preventing and/or combating a decrease in vascularization of the skin and/or of the mucous membranes and/or of the scalp and/or for improving it, in particular for improving the structure of the capillaries of the skin and/or of the mucous membranes and/or of the scalp, which are in particular cutaneous, in particular by increasing the expression of claudin 5 in the skin, for improving the morphogenesis of the epithelium, of the skin and/or of the mucous membranes and/or of the scalp, preferentially the epidermis, for restoring the epithelial architecture of the skin and/or of the mucous membranes and/or of the scalp, preferentially the epidermal architecture, in particular of skin and/or of mucous membranes and/or of the scalp having undergone ageing, in particular chronobiological ageing, for improving the complexion of the skin and/or of the mucous membranes, especially making it uniform, in particular by increasing the expression of claudin 5 in the skin, for improving the firmness and/or the density of the skin and/or of the mucous membranes and/or of the scalp, and/or for combating a decrease in the thickness of the epithelium of the skin and/or of the mucous membranes and/or of the scalp, preferentially the epidermis, and/or for increasing the thickness of the epithelium of the skin and/or of the mucous membranes and/or of the scalp, preferentially the epidermis.

In one particular embodiment, the purpose of the use according to the present invention is to improve the complexion of the skin, advantageously by eliminating red patches and/or by making the complexion uniform and/or by giving it a luminous, radiant, healthy and/or nourished appearance, a well-looking effect and/or a pinkish radiance.

Indeed, the radiance of the complexion generally reflects a healthy condition of the skin. Numerous intrinsic or extrinsic factors can cause a non-uniform muddy complexion. Among the factors which affect the radiance of the complexion of the skin, mention may be made of stress, fatigue, hormonal changes, dehydration of the epithelium, preferentially the epidermis, polluting agents and chronobiological and photobiological ageing. These factors tend to make the complexion muddy, and to make it non-uniform, dull, waxy, or even sickly. The expression of perlecan and of dystroglycan has an impact on the microvascular pathway, thereby making it possible to nourish the skin better and to detoxify it better and therefore to give it a nourished and healthy appearance. In addition, the color of the skin is influenced by the microcirculation: when reaching the microvessels, light comes into contact with the red blood cells which specifically absorb in the green range. Red is reflected at the surface, giving the skin a pinkish hue and therefore a pinkish radiance. Thus, increasing the expression of claudin 5 makes it possible to improve the radiance of the complexion. The radiance of the complexion also depends on the reflecting capacity of the skin. This reflecting capacity is influenced by the texture of the skin. Skin which has a softer, more flexible texture will therefore have a better aspect. The restoration of the epidermal architecture and the improvement of the morphogenesis of the epithelium, preferentially the epidermis obtained, by virtue of the stimulation of the expression of perlecan and/or of dystroglycan will also therefore make it possible to improve the skin complexion.

The improvement in the radiance or glow of the complexion can in particular be measured by an objective instrumental method. This in vivo method of measurement consists in taking high-resolution photographs, with cross-polarized light, of the face of volunteers taken at 45° before and after application of the product tested. On the basis of these digital photographs, an image analysis makes it possible to extract and to quantify specific parameters (for example: L*, a*, b*, C, h°) related to the color, radiance, uniformity and texture of the skin.

Likewise, the gloss can in particular be measured according to this method on the basis of high-resolution photographs, with cross-polarized and parallel-polarized light, of the face of volunteers taken at 45° before and after application of the product tested. On the basis of these digital photographs, an image analysis makes it possible to extract and to quantify specific parameters related to the gloss, such as the specular gloss and the contrast gloss.

In another particular embodiment, the purpose of the use according to the present invention is to prevent and/or combat ageing of the skin and/or of the mucous membranes and/or of the scalp, in particular chronobiological or photobiological ageing, by decreasing or suppressing the wrinkles and/or fine lines, in particular for mature skin and/or skin showing the first signs of ageing.

For the purposes of the present invention, the term “mature skin” is intended to mean skin of men or women who are at least 50 years old, advantageously skin of menopausal women.

For the purposes of the present invention, the term “skin showing the first signs of ageing” is intended to mean skin of men or women who are between 30 and 40 years old, advantageously skin showing the first expression wrinkles.

In yet another particular embodiment, the purpose of the use according to the present invention is to prevent and/or combat a decrease in the homeostasis of the skin and/or of the mucous membranes and/or of the scalp and/or to improve it, in particular in the epidermis.

Homeostasis, especially cutaneous homeostatis, and in particular epidermal homeostatis, results from a finely regulated balance between the processes of proliferation and differentiation of the cells of the skin and in particular of the keratinocytes. These proliferation and differentiation processes participate in the renewal and/or in the regeneration of the skin and result in the maintaining of a constant thickness of the skin, and in particular of a constant thickness of the epithelium, preferentially the epidermis. This homeostasis also participates in maintaining the mechanical properties of the skin, of the mucous membranes and of the scalp.

However, this cutaneous homeostatis can be impaired by certain physiological factors (age, menopause, hormones, stress, etc.) and extrinsic factors (polluting agents, etc.). The regenerative potential of the epithelium, in particular the epidermis, becomes lower: the cells of the basal layer divide less actively, resulting in particular in a slowing down and/or a reduction of epidermal renewal. Consequently, cell renewal no longer compensates for the loss of cells eliminated at the surface, resulting in atrophy of the epithelium, in particular the epidermis, and/or a decrease in the thickness of the skin and/or of the mucous membranes and/or of the scalp and/or a loss of density and/or of firmness of the skin and/or of the mucous membranes and/or of the scalp. This phenomenon can be accentuated by the menopause. The hormonal deficiencies associated with the menopause are accompanied in particular by a decrease in metabolic activity, which could result in a decrease in keratinocyte proliferation and an increase in epidermal differentiation. The use according to the present invention therefore makes it possible to promote homeostasis in order to maintain and/or increase the thickness of the skin and/or of the mucous membranes and/or of the scalp and thus to maintain and/or improve the mechanical properties of the skin and/or of the mucous membranes and/or of the scalp and/or improve the firmness and/or the density of the skin and/or of the mucous membranes and/or of the scalp, in particular in menopausal women.

The increase in the thickness of the epithelium, preferentially the epidermis, can in particular be evaluated ex vivo on a skin and/or mucous membrane and/or scalp biopsy model under survival conditions. The epithelium, preferentially the epidermis, is measured at the beginning of the experiment before the application of the test product and at the end of the test period in the presence of the test product, for example 4 days, preferentially 7 days. The thickness of the epithelium, preferentially the epidermis, is said to be increased if the measure after application of the product is greater than or equal to 5% at the end of the test period, preferentially greater than or equal to 10%.

Advantageously, the concerned area of the skin, in particular of the human being, to which the Polygonum bistorta extract according to the invention or a cosmetic or dermatological composition comprising it is applied is chosen from the face, the neck, the neckline, the bust and/or the hands, and quite particularly the nasal grooves and/or the periorbital area, in particular on the dark circles and the crows feet and/or the outline of the lips and/or the forehead. However, the extract may also be applied to the body, in particular to the stomach, the thighs, the hips, the buttocks and/or the waist, these being areas of the body which can show a loss of firmness and/or density.

According to the invention, the Polygonum bistorta plant extract according to the invention is used alone or in a cosmetic or dermatological composition, at a concentration of between 1×10⁻⁴ and 10% by weight, and advantageously between 1×10⁻⁴ and 5% and more particularly between 1×10⁻³ and 3% by weight relative to the weight of the total composition.

Advantageously, the Polygonum bistorta extract is present in a cosmetic or dermatological composition according to the invention in a content of between 1×10⁻⁴ and 10% by weight relative to the total weight of the composition, preferentially between 1×10⁻⁴ and 5% by weight relative to the total weight of the composition, advantageously between 1×10⁻³ and 3% by weight relative to the total weight of the composition, in particular between 0.001% and 0.1% by weight relative to the total weight of the composition. Advantageously, the Polygonum bistorta extract contains an amount of resveratrol and/or trans-stilbene (C₁₄H₁₂, MW 180.24 g/mol) and/or of resveratrol derivatives in particular in the form of resveratrol oligomers and/or resveratrol analogs, such as rhapontin (of formula C₂₁H₂₄O₉, MW 420.14 g/mol), deoxyrhapontin (C₂₁H₂₄O₈, MW 404.14 g/mol), epsilon-viniferin, acetates, formic esters, 3,4′,5-trihydroxystilbene-3-O-beta-D-glucopyranoside (C₂₀H₂₂O₈, MW 390.13 g/mol), and/or resveratrol glucosides, of less than 0.001%, preferentially less than 0.0001% by weight relative to the total weight of the composition.

Preferentially, the composition according to the invention contains an amount of resveratrol and/or of trans-stilbene (C₁₄H₁₂, MW 180.24 g/mol) and/or of resveratrol derivatives in particular in the form of resveratrol oligomers and/or resveratrol analogs, such as rhapontin (of formula C₂₁H₂₄O₉, MW 420.14 g/mol), deoxyrhapontin (C₂₁H₂₄O₈, MW 404.14 g/mol), epsilon-viniferin, acetates, formic esters and/or 3,4′,5-trihydroxystilbene-3-O-beta-D-glucopyranoside (C₂₀H₂₂O₈, MW 390.13 g/mol), and/or resveratrol glucosides, of less than 0.001%, preferentially less than 0.0001% by weight relative to the total weight of the composition. Even more preferentially, the composition according to the invention does not contain resveratrol and/or trans-stilbene (C₁₄H₁₂, MW 180.24 g/mol) and/or resveratrol derivatives in particular in the form of resveratrol oligomers and/or resveratrol analogs, such as rhapontin (of formula C₂₁H₂₄O₉, MW 420.14 g/mol), deoxyrhapontin (C₂₁H₂₄O₈, MW 404.14 g/mol), epsilon-viniferin, acetates, formic esters, and/or 3,4′,5-trihydroxystilbene-3-O-beta-D-glucopyranoside (C₂₀H₂₂O₈, MW 390.13 g/mol).

In one embodiment according to the present invention, the Polygonum bistorta extract is used for the production of a dermatological composition for the care and/or the dermatological treatment of rosacea, telangiectasias, chapping, and/or for the care and/or treatment of pathological conditions of the buccal and/or ocular mucous membranes, in particular for the care and/or treatment of pathological conditions of the buccal mucous membranes involving a loss of firmness and/or of density at the level of the buccal mucous membrane and/or for improving the buccal vascular structure and/or for the care and/or treatment of pathological conditions of the ocular mucous membranes involving a loss of firmness and/or of density at the level of the ocular mucous membrane and/or for improving the ocular vascular structure.

In another embodiment according to the present invention, the Polygonum bistorta extract according to the invention is present in a cosmetic or dermatological composition comprising a cosmetically or dermatologically acceptable excipient.

This cosmetic or dermatological composition is intended for topical application.

The terms “cosmetically or dermatologically acceptable excipients”, used herein, signify that the composition or the constituents of said composition are suitable for use in contact with human skin and/or mucous membrane and/or the human scalp without undue toxicity, incompatibility, instability, allergic response or equivalents thereof.

The cosmetic or dermatological compositions according to the invention thus contain a cosmetically or dermatologically acceptable excipient in addition to the extract according to the invention. This excipient is, for example, at least one compound chosen from the group consisting of preservatives, emollients, emulsifiers, surfactants, moisturizers, thickeners, conditioners, matting agents, stabilizers, antioxidants, texturing agents, gloss agents, film-forming agents, solubilizing agents, pigments, dyes, fragrances and sunscreens. These excipients are preferably chosen from the group consisting of amino acids and derivatives thereof, polyglycerols, esters, cellulose polymers and derivatives, lanolin derivatives, phospholipids, lactoferrins, lactoperoxidases, sucrose-based stabilizers, vitamin E and derivatives thereof, natural and synthetic waxes, vegetable oils, triglycerides, unsaponifiable compounds, phytosterols, plant esters, silicones and derivatives thereof, protein hydrolysates, jojoba oil and derivatives thereof, lipo/water-soluble esters, betaines, aminoxides, plant extracts, sucrose esters, titanium dioxides, glycines, and parabens, and more preferably from the group consisting of steareth-2, steareth-21, glycol-15 stearyl ether, cetearyl alcohol, phenoxyethanol, methylparaben, ethylparaben, propylparaben, butylparaben, butylene glycol, caprylyl glycol, natural tocopherols, glycerin, dihydroxycetyl sodium phosphate, isopropyl hydroxycetyl ether, glycol stearate, triisononanoin, octyl cocoate, polyacrylamide, isoparaffin, laureth-7, a carbomer, propylene glycol, hexylene glycol, glycerol, bisabolol, a dimethicone, sodium hydroxide, PEG 30-dipolyhydroxystearate, capric/caprylic triglycerides, cetearyl octanoate, dibutyl adipate, grapeseed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG 8, beeswax, hydrogenated palm kernel oil glycerides, lanolin oil, sesame oil, cetyl lactate, lanolin alcohol, castor oil, titanium dioxide, lactose, sucrose, low-density polyethylene, and an isotonic saline solution.

The cosmetic composition according to the invention may be chosen from an aqueous or oily solution, a cream or an aqueous gel or an oily gel, in particular a shower gel, a shampoo; a milk; an emulsion, a microemulsion or a nanoemulsion, which is in particular oil-in-water or water-in-oil or multiple or silicone-based; a mask; a serum; a lotion; a liquid soap; a dermatological bar; an ointment; a foam; a patch; an anhydrous product, which is preferably liquid, paste or solid, for example in the form of makeup powders, of a wand or of a stick, in particular in the form of a lipstick. Advantageously, it is a cream or a serum, in particular an eye or lip liner.

The compositions according to the invention are more particularly applied to the face, preferentially daily, preferentially once or twice a day, preferentially in the morning and/or the evening.

Advantageously, the cosmetic compositions according to the invention contain other ingredients of interest, in particular cosmetic interest, preferentially agents which have similar properties. Preferentially, these are the conventional ingredients of anti-ageing compositions and/or compositions which improve the density and/or the firmness of the skin and/or of the mucous membranes and/or which improve the complexion of the skin and/or cutaneous homeostatis, in particular those chosen from filling agents, tensioning agents, moisturizing agents, and agents for stimulating extracellular matrix molecules.

The cosmetic compositions according to the invention may also contain cosmetic active ingredients which produce a supplementary or optionally synergistic effect, such as moisturizing active agents, anti-ageing active agents, free-radical scavenging active agents, fibroblast growth factor (FGF) protecting agents, agents for stimulating fibroblast activity and/or proliferation and/or spring waters. They may thus be, for example, skin-coloring or propigmenting agents, NO-synthase inhibitors, antiseborrheic agents for oily-skin care, agents for stimulating the synthesis of dermal or epidermal macromolecules, in particular of the extracellular matrix, and/or preventing degradation thereof, for a synergistic or supplementary effect, agents for stimulating fibroblasts or keratinocyte proliferation and/or keratinocyte differentiation, for a synergistic or supplementary effect, antimicrobial agents, tensioning agents, antipollution agents or free-radical scavengers, soothing, calming or relaxing agents, agents which act on the microcirculation to improve the radiance of the complexion, in particular of the face, for a synergistic or supplementary effect, photoprotective agents, healing agents, slimming agents, anti-ageing agents for a synergistic or supplementary effect or optionally moisturizing agents and/or agents for reinforcing the epidermal barrier.

The moisturizing, emollient or humectant active agents can reinforce the barrier function and reduce insensible water losses and/or increase the water content of the skin and/or of the mucous membranes or stimulate the secretory activity of the sebaceous glands and/or stimulate the synthesis of aquaporin in order to improve the circulation of water in the cells. By way of nonlimiting example, mention may be made of the following active agents: serine, urea and its derivatives, the products sold under the name Marine Filling Spheres™, Advanced Moisturizing Complex™, Hyaluronic Filling Spheres™, Vegetal filling spheres™, Osmogelline™, Micropatch™, alkylcelluloses, lecithins, sphingoid-based compounds, ceramides, phospholipids, cholesterol and its derivatives, glycosphingolipids, phytosterols (stigmasterol and beta-sitosterol, campesterol), essential fatty acids, 1,2-diacylglycerol, 4-chromanone, pentacyclic triterpenes such as ursolic acid, petroleum jelly, lanolin, sugars, in particular trehalose and its derivatives, rhamnose, fructose, maltose, lactose, erythritol, mannitol, D-xylose and glucose, adenosine and its derivatives, sorbitol, polyhydric alcohols, advantageously C₂-C₆, and more advantageously C₃-C₆ polyhydric alcohols, such as glycerin, propylene glycol, dipropylene glycol, diglycerin, polyglycerin, and mixtures thereof, glycerol and its derivatives, glyceryl polyacrylate, sodium lactate, pentanediol, serine, lactic acids, AHAs, BHAs, sodium pidolate, xylitol, sodium lactate, ectoin and its derivatives, chitosan and its derivatives, collagen, plankton, steroidal derivatives (including DHEA, its 7-oxidized and/or 17-alkylated derivatives and sapogenins), methyl dihydrojasmonate, vitamin D and its derivatives, a Malva sylvestres extract or a Centella asiatica extract, acrylic acid homopolymers, beta-glucan and in particular sodium carboxymethyl beta-glucan, a C-glycoside derivative such as those described in application WO 02/051828, a musk rose oil, an extract of the microalga Prophyridium cruentum enriched with zinc, sold by Vincience under the name Algualane Zinc™, arginine, the acetyl hexapeptide sold by Lipotech under the name Diffuporine™, the Viola tricolor hydrolysate sold by Silab under the name Aquaphyline™.

The active agent may also be chosen from anti-ageing agents, i.e. agents having in particular a restructuring effect on the skin barrier, agents for preventing and/or reducing the glycation of skin proteins, in particular dermal proteins, such as collagen, active agents for stimulating the energy metabolism of cells, and mixtures thereof, an agent with an overall anti-ageing action, in particular niacinamide or vitamin B3 and derivatives. The agent having a restructuring effect on the skin barrier may be chosen from one of the yeast extracts, such as Relipidium™ from BASF Beauty Care Solutions France SAS, sphingosines, such as salicyloyl sphingosine, a mixture of xylitol, xylityl polyglycoside and xylitan, extracts of Solanaceae, such as Lipidessence™ from BASF Beauty Care Solutions France SAS and mixtures thereof. Mention may also in particular be made of ceramides, sphingoid-based compounds, glycosphingolipids, phospholipids, cholesterol and its derivatives, phytosterols, essential fatty acids, diacylglycerol, 4-chromanone and chromone derivatives and mixtures thereof, vitamin B5 or pantothenate and derivatives.

The active agent for stimulating the energy metabolism of cells may, for example, be chosen from biotin, a mixture of sodium salts, manganese salts, zinc salts and magnesium salts of pyrrolidonecarboxylic acid, a mixture of zinc gluconate, copper gluconate and magnesium gluconate, and mixtures thereof.

The antiseborrheic agent in the composition according to the invention may be a 5α-reductase inhibitor, such as retinoids, sarcosine, zinc salts, in particular zinc gluconate, zinc salicylate, azelaic acid, and/or derivatives thereof, and/or mixtures thereof, and an Orthosiphon stamineus extract sold under the name MAT XS™ bright by BASF Beauty Care Solutions France SAS.

The composition may also contain a sebum-absorbing agent, in particular a talc and/or an absorbent polymer, an antibacterial agent, in particular those described in patent application FR2863893, and in particular a Boldo extract, such an extract being in particular sold by the applicant under the name Betapur™, a comedolytic agent, in particular retinoic acid or a derivative thereof such as isotretinoin, adapalene and/or 13-cis-retinoic acid and benzoyl peroxide, a local antibiotic agent, in particular erythromycin and/or clindamycin phosphate and mixtures thereof.

Among the active agents for stimulating the synthesis of dermal macromolecules or preventing the degradation thereof, mention may be made of those which act as:

an agent for stimulating fibronectin synthesis, in particular a corn extract, such an extract being in particular sold by the applicant under the name Deliner™, and the palmitoyl pentapeptide sold by the company Sederma under the trade name Matrixil™,

an agent for protecting extracellular matrix fibroblast growth factor (FGF2) against degradation thereof and/or denaturation thereof, in particular a Hibiscus abelmoscus extract as described in the patent application in the name of the applicant filed under number FR0654316 and/or an agent for stimulating fibroblasts growth, for example a fermented soya extract containing peptides, known under the name Phytokine™ sold by the applicant and also described in patent application EP 1 119 344 B1 (Laboratoires Expanscience), and preferentially a combination of these two extracts;

an agent for stimulating laminin synthesis, in particular an extract of malt modified by biotechnology, such an extract being in particular sold by the applicant under the name Basaline™;

an agent for stimulating the expression and/or activity of hyaluronan synthase 2 (HAS2), such as the plant extracts described in patent application FR 2 893 252 A1 and in particular an aqueous extract of Galanga (Alpinia galanga);

an agent for stimulating the synthesis of lysyl oxidase like (LOXL), such as a Geophila cordifolia extract and those described in patent application FR2855968, in particular a dill extract;

an agent for stimulating intracellular ATP synthesis, in particular an extract of the alga Laminaria digitata;

an active agent for stimulating glycosaminoglycan synthesis, such as the product of milk fermentation;

a collagen-stimulating active agent, such as retinol and/or vitamin C;

an active agent which inhibits metalloproteinases (MMPs), such as more particularly MMP 1, 2, 3 and 9, such as retinoids and derivatives, oligopeptides and lipopeptides, lipoamino acids, the malt extract sold by BASF Beauty Care Solutions France under the trade name Collalift™, the hydrolyzed potato extract sold under the name Extracellium™ by BASF Beauty Care Solutions France SAS; lycopene; isoflavones, quercetin, kaempferol and apigenin.

The agents for stimulating keratinocyte proliferation that can be used in the composition according to the invention comprise in particular retinoids, such as retinal and its esters, including retinyl palmitate and phloroglucinol. The agents for stimulating keratinocyte differentiation comprise, for example, minerals such as calcium and lignans such as secoisolariciresinol, and also the Achillea millefollium extract sold under the name Neurobiox™ by BASF Beauty Care Solutions France.

The antimicrobial agents that may be used in the composition according to the invention can in particular be chosen from 2,4,4′-trichloro-2′-hydroxy diphenyl ether (or triclosan), 3,4,4′-trichlorobanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, octoxyglycerin, octanoylglycine, caprylyl glycol, 10-hydroxy-2-decanoic acid, farnesol, phytosphingosines, and mixtures thereof.

Among the tensioning agents that can be used in the composition according to the present invention, mention may in particular be made of synthetic polymers, such as polyurethane latexes or acrylic latexes; polymers of natural origin, in particular polyholosides in the form of starch or in the form of carrageenans, alginates, agars, gellans, cellulose-based polymers and pectins; soya vegetable proteins and protein hydrolysates; mixed silicates; wax microparticles; colloidal particles of inorganic filler, chosen for example from silica and silica-alumina composites; and also mixtures thereof.

The composition may comprise “antipollution” agents, in particular ozone scavengers represented, for example, by vitamin C and its derivatives, including ascorbyl glucoside; phenols and polyphenols, in particular tannins, ellagic acid and tannic acid; epigallocatechin and natural extracts containing same, in particular green tea extracts; anthocyans; phenol acids, stilbenes; active agents which are mono- or polycyclic aromatic compound scavengers, tannins such as ellagic acid and indole derivatives and/or active agents which trap heavy metals, such as EDTA, active agents which are free-radical scavengers, such as vitamin E and its derivatives such as tocopheryl acetate; bioflavonoids; coenzyme Q10 or ubiquinone.

As soothing agents that can be used in the composition according to the invention, mention may be made of: pentacyclic triterpenes, ursolic acid and its salts, oleanolic acid and its salts, betulinic acid and its salts, salicylic acid salts and in particular zinc salicylate, bisabolol, allantoin, unsaturated omega-3 oils, cortisone, hydrocortisone, indomethacin and betamethasone, anti-inflammatory active agents, and in particular those described in application FR2847267, in particular the Pueraria lobata root extract sold under the name Inhipase™ by BASF Beauty Care Solutions France SAS, Theobroma cacao extracts.

The active ingredients which act on the microcirculation (vasoprotectors or vasodilators) may be chosen from flavonoids, ruscogenins, nicotinates and essential oils.

The photoprotective active agent or UVA- and/or UVB-screening agent ingredients that can be used according to the present invention are in particular protoprotective agents which are active in the UVA and/or UVB range, such as para-aminobenzoic acid derivatives, in particular Uvinul P25™ sold by BASF, salicylic derivatives, in particular homosalate alone or in association with titanium oxides, dibenzoylmethane derivatives, cinnamic derivatives, diphenyl acrylate derivatives, including Octocrylene sold in particular under the trade name Uvinul N539™ by BASF, benzophenone derivatives, in particular Benzophenone-1 sold in particular under the trade name Uvinul 400™ by BASF, benzylidenecamphor derivatives, benzimidazole derivatives, triazine derivatives, including ethylhexyl triazone sold in particular under the trade name Uvinul T150™ by BASF, benzotriazole derivatives, anthranilic derivatives, imidazoline derivatives, benzalmalonate derivatives, 4,4-diarylbutadiene derivatives, and mixtures thereof.

The active agents which provide an effect of well-being, such as those which mimic the effects of beta-endorphins for improving the barrier function of the skin, such as those mentioned in patent application U.S. 2006069032; active agents stimulating the synthesis of beta-endorphins, such as an extract of the plant Tephrosia purpurea.

The slimming active agents may in particular be chosen from: agents which inhibit lipoprotein lipase, such as those described in patent U.S. 2003086949 (Coletica) and in particular an extract of Peruvian liana (Uncaria tomentosa); draining active agents, in particular hesperitin laurate (Flavagrum™), or quercitin caprylate (Flavenger™); agents which inhibit the enzyme phosphodiestarase, agents which activate adenylate cyclase, cAMP and/or agents capable of trapping spermine and/or spermidine. By way of example of these active agents, mention may be made of a Coleus forskohlii root extract, a Cecropia obtusa extract, a Uva lactuca extract, caffeine, forskolin, theophylline, theobromine, and/or derivatives thereof, a hydrolyzed kappa carrageenan product called Slimexcess™ sold by BASF Beauty Care Solutions France SAS, and/or mixtures thereof.

In one particular embodiment, the cosmetic composition according to the present invention does not contain any depigmenting and/or anti-tyrosinase and/or melanogenesis-inhibiting agent.

Numerous cosmetically active ingredients are known by those skilled in the art to improve the health and/or the physical appearance of the skin and/or the mucous membranes and/or the scalp. Those skilled in the art know how to formulate cosmetic compositions in order to obtain the best effects.

Furthermore, the compounds described in the present invention can have a synergistic effect when they are combined with one another. These combinations are also covered by the present invention. The CTFA Cosmetic Ingredient Handbook, Second Edition (1992) describes various cosmetic and pharmaceutical ingredients commonly used in the cosmetics industry, which are in particular suitable for topical use. Examples of these classes of ingredients include, without being limited thereto, the following compounds: abrasive, absorbents, compound for esthetic purposes, such as fragrances; pigments; dyes; essential oils; astringents such as clove oil, menthol, camphor, eucalyptus oil, eugenol, menthyl lactate, witch hazel distillate; anti-acne agents; antiflocculants; antifoams; antimicrobial agents such as iodopropyl butylcarbamate; antioxidants such as ascorbic acid; binders; biological additives; buffers; swelling agents; chelating agents; additives; biocidal agents; denaturing agents; thickeners; and vitamins; film-forming materials; polymers; opacifiers; pH modifiers; reducing agents; conditioning agents such as humectants, and derivatives or equivalents thereof.

In yet another particular embodiment of the present invention, the Polygonum bistorta extract according to the invention, preferentially obtained by aqueous extraction, is dissolved in a solvent, in particular a polar solvent, such as water, an alcohol, a polyol, a glycol, or a mixture thereof, preferentially an aqueous-glycolic mixture, more preferentially containing a glycol chosen from caprylyl glycol, hexylene glycol and mixtures thereof.

Particularly advantageously, the extract according to the invention is solubilized in an aqueous solution comprising hexylene glycol, caprylyl glycol or a mixture thereof, advantageously hexylene glycol and caprylyl glycol.

Advantageously, the aqueous solution in which the Polygonum bistorta extract according to the invention is solubilized comprises hexylene glycol, in particular between 0.1% and 10% by weight of hexylene glycol relative to the total weight of the aqueous solution, more particularly between 1% and 5% by weight of hexylene glycol relative to the total weight of the aqueous solution.

In particular, the aqueous solution in which the Polygonum bistorta extract according to the invention is solubilized comprises caprylyl glycol, in particular between 0.01% and 5% by weight of caprylyl glycol relative to the total weight of the aqueous solution, more particularly between 0.1% and 1% by weight of caprylyl glycol relative to the total weight of the aqueous solution. Particularly advantageously, the aqueous solution in which the Polygonum bistorta extract according to the invention is solubilized comprises hexylene glycol and caprylyl glycol, in particular in the proportions indicated above.

Advantageously, the Polygonum bistorta extract according to the invention, preferentially obtained by aqueous extraction, is solubilized in the aqueous solution in a content of between 0.1% and 10% by weight relative to the total weight of the aqueous solution, in particular of between 1% and 5% by weight relative to the total weight of the aqueous solution. In particular, this aqueous solution comprises hexylene glycol and caprylyl glycol, advantageously in the proportions indicated above for these constituents.

The aqueous solution in which the extract according to the invention is solubilized may comprise a thickener and/or structuring agent such as xanthan gum, advantageously in a content of between 0.01% and 5% by weight relative to the total weight of the aqueous solution, in particular between 0.1% and 1% by weight relative to the total weight of the aqueous solution.

The present invention also relates to a cosmetic care method, characterized in that it comprises the application, to at least one concerned area of healthy skin and/or healthy mucous membrane and/or healthy scalp, in particular of a human being, of a Polygonum bistorta extract or of a cosmetic composition comprising such an extract for stimulating the expression of perlecan and/or of dystroglycan, in particular in the extracellular matrix and/or in the epithelial basement membrane, in particular the dermoepidermal junction.

Advantageously, this cosmetic care method is for preventing and/or combating ageing of the skin and/or of the mucous membranes and/or of the scalp, in particular chronobiological and/or photobiological ageing, for preventing and/or combating a decrease in homeostasis of the skin and/or of the mucous membranes and/or of the scalp and/or for improving it, in particular in the epidermis, for reinforcing the epithelial basement membrane of the skin and/or of the mucous membranes and/or of the scalp, preferentially the dermoepidermal junction, for improving keratinocyte proliferation and/or differentiation, especially at the epidermal level, in particular associated with ageing of the skin and/or of the mucous membranes and/or of the scalp, for preventing and/or combating a decrease in vascularization of the skin and/or of the mucous membranes and/or of the scalp and/or for improving it, in particular for improving the structure of the capillaries of the skin and/or of the mucous membranes and/or of the scalp, which are in particular cutaneous, for improving the morphogenesis of the epithelium, of the skin and/or of the mucous membranes and/or of the scalp, preferentially the epidermis, for restoring the epithelial architecture of the skin and/or of the mucous membranes and/or of the scalp, preferentially the epidermal architecture, in particular of skin and/or of mucous membranes and/or of the scalp having undergone ageing, in particular chronobiological ageing, for improving the complexion of the skin and/or of the mucous membranes, especially making it uniform, for improving the firmness and/or the density of the skin and/or of the mucous membranes and/or of the scalp, and/or for combating a decrease in the thickness of the epithelium of the skin and/or of the mucous membranes and/or of the scalp, preferentially the epidermis, and/or for increasing the thickness of the epithelium of the skin and/or of the mucous membranes and/or of the scalp, preferentially the epidermis.

Other purposes, characteristics and advantages of the invention will emerge clearly to those skilled in the art following the reading of the explanatory description which makes reference to examples which are given only by way of illustration and which could not in any way limit the scope of the invention. The examples are an integral part of the present invention and any characteristic which appears novel over any prior art on the basis of the description taken as a whole, including the examples, is an integral part of the invention with respect to its function and with respect to its general nature.

Thus, each example has a general scope.

Furthermore, in the examples, and unless otherwise indicated, the temperature is expressed in degrees Celsius, and the pressure is atmospheric pressure.

### FIGURES

FIG. 1 represents the change in the level of expression of perlecan measured in a biopsy from a 50-year-old donor, in culture without treatment (control) or with treatment (Extract at 0.5%) at 0 day (T0) and 7 days (T7) of culture according to the test described in example 6.

### EXAMPLE 1

### Preparation of Polygonum bistorta Extract According to the Invention by Aqueous Extraction

a) Polygonum bistorta roots are milled and then macerated in water for 2 hours at ambient temperature, i.e. between 18 and 25° C., in this case at approximately 20° C., the content of milled Polygonum bistorta roots being 1% by weight relative to the total plant/water weight.

The insoluble materials are separated by filtration at 0.45 μm and the liquid which contains the aqueous extract according to the invention is recovered. This extract was tested at various dosages in the final culture medium in the following examples 2, 4, 5 and 6.

The extract thus obtained contains neither resveratrol, nor trans-stilbene (C₁₄H₁₂, MW 180.24 g/mol), nor rhapontin (of formula C₂₁H₂₄O₉, MW 420.14 g/mol), nor deoxyrhapontin (C₂₁h₂₄O₈, MW 404.14 g/mol), nor epsilon-viniferin, nor 3,4′,5-trihydroxystilbene-3-O-beta-D-glucopyranoside (C₂₀h₂₂O₈, MW 390.13 g/mol).

This extract can also be formulated in the form of a cosmetic ingredient as presented in example 7.

b) The Polygonum bistorta roots are milled and then macerated in water at 1% (w/w), at a temperature preferentially between 0 and 20° C., preferentially at 4° C.

The maceration time is advantageously between 30 min and 24 hours, with stirring, in this case 16 hours.

The solution is centrifuged, preferentially for 10 min at 8000 rpm and the supernatant is recovered. The supernatant is ultrafiltered on filters at various cut-off thresholds and in particular at 0.22 μm.

The extract thus obtained can be used directly in liquid form. It was used at various dosages in example 3.

c) The Polygonum bistorta roots are milled and then macerated in a water/butylene glycol mixture at 75%/25% at a temperature preferentially between 0 and 20° C., in this case at 4° C. The maceration time is advantageously between 30 min and 24 hours, with stirring, in this case 10 hours.

The solution is centrifuged, preferentially for 10 min at 8000 rpm and the supernatant is recovered. The supernatant is ultrafiltered on filters at various cut-off thresholds and in particular at 0.45 μm.

The extract thus obtained is then dried, in particular on a maltodextrin support, and then resolubilized in water at 1% (w/w).

### EXAMPLE 2

### Effect of a Polygonum bistorta Extract According to the Invention on the Expression of Perlecan in Keratinocytes

A fluoroimmunoassay (FIA) test was carried out and consists in revealing the antigen of interest, in this case perlecan, by fluorescence. This method is semi-quantitative, highly sensitive and reproducible, and has the advantage of detecting the protein of interest in its native form in its environment, without a denaturation process. The keratinocytes from an abdominal skin biopsy from a 50-year-old donor are extracted and attached to the bottom of the wells at a density of 5000 cells per well, and grow for 3 days in a complete medium, i.e. a medium containing fœtal calf serum (FCS), then for 16 hours in a defined medium without FCS. They are then cultured for 48 hours either with the extract obtained in example 1a), tested at various dosages as percentage by weight in the final culture medium, or without the extract, termed control. The cells are then washed in phosphate buffered saline (PBS), before being fixed, permeabilized and unmasked. Before applying the (anti-perlecan) primary antibody for 90 minutes, the cells are saturated with BSA (bovine serum albumin) at 1% for 1 hour. After washes in PBS buffer, the secondary antibody linked to the fluorocrome FITC (fluorescein isothiocyanate) is incubated for 2 hours. The cells are then washed in PBS buffer, and then solubilized with 20 mM of ammonium hydroxide and 0.5% of 100% triton. The fluorescence is then read on a spectrofluorimeter with the appropriate filters. The results are collated in table 1 below. The experiment is carried out 12 times (n=12). The values in the table represent the values as percentage relative to the nontreated control cells. The values represent the mean of several experiments on various batches of extracts. “Mean” denotes the mean and “SD” denotes the standard deviation.

TABLE 1
percentage expression of perlecan in keratinocytes
as a function of the dose of extract used

| | Mean | SD |
|---|---|---|
| Control | 100 | 1 |
| 0.1% Polygonum bistorta extract | 157.5 | 24.5 |
| 0.5% Polygonum bistorta extract | 266.9 | 40.5 |
| 1% Polygonum bistorta extract | 175.3 | 45.1 |

Conclusions:

The extract according to the invention induces a significant increase in perlecan protein expression in the keratinocytes. The extract according to the invention therefore induces an improvement in the structural cohesion of the epithelium, preferentially the epidermis.

### EXAMPLE 3

### Effect of a Polygonum bistorta Extract According to the Invention on the Expression of Perlecan in Endothelial Cells

The technique is the same as in example 2, except that it is carried out on endothelial cells extracted from abdominal skin biopsy from 50-year-old or 30-year-old or neonatal donors.

The results are collated in tables 2, 2a, 3 and 4 below; “Mean” denotes the mean and “SD” denotes the standard deviation.

TABLE 2
percentage expression of perlecan in the endothelial cells of a
50-year-old donor as a function of the dose of extract used. n = 12; the
Polygonum bistorta extract is obtained according to example 1b) tested
at various dosages as percentage by weight in the final culture medium

| | Mean | SD |
|---|---|---|
| Control | 100 | 4.2 |
| 0.1% Polygonum bistorta extract | 127 | 2.9 |
| 0.5% Polygonum bistorta extract | 122 | 7.6 |

TABLE 2a
percentage expression of perlecan in the endothelial cells of a
50-year-old donor as a function of the dose of extract used. n = 12; the
Polygonum bistorta extract is obtained according to example 1a) tested
at various dosages as percentage by weight in the final culture medium

| | Mean | SD |
|---|---|---|
| Control | 100 | 25 |
| 0.1% Polygonum bistorta extract | 166 | 16 |
| 0.5% Polygonum bistorta extract | 200 | 28 |
| 1% Polygonum bistorta extract | 209 | 31 |

TABLE 3
percentage expression of perlecan in the endothelial cells of a
30-year-old donor as a function of the active agent used. n = 12; the
Polygonum bistorta extract is obtained according to example 1b) tested
at various dosages as percentage by weight in the final culture medium

| | Mean | SD |
|---|---|---|
| Control, nontreated | 100 | 10 |
| 0.1% Polygonum bistorta aqueous extract | 113 | 4 |
| 0.5% Polygonum bistorta aqueous extract | 132 | 15 |

TABLE 4
percentage expression of perlecan in the endothelial cells of a
neonatal donor as a function of the dose of extract used. n = 6; the
Polygonum bistorta extract is obtained according to example 1b) tested
at various dosages as percentage by weight in the final culture medium

| | Mean | SD |
|---|---|---|
| Control | 100 | 6 |
| 0.1% Polygonum bistorta extract | 127 | 6 |
| 0.5% Polygonum bistorta extract | 145 | 5 |

Conclusions:

The extract according to the invention significantly increased, at the doses tested, the perlecan protein expression in the endothelial cells, thereby attesting to its properties in improving the microvascular structural cohesion. This increase is observed regardless of the age of the donors. This example also demonstrates the better effectiveness of the extract according to example 1a).

### EXAMPLE 4

### Effect of a Polygonum bistorta Extract According to the Invention on Expression of Dystroglycan in Keratinocytes

The technique is the same as in example 2, except that the antigen of interest is in this case dystroglycan and the antibody used is an anti-dystroglycan.

The results are collated in table 5 hereinafter; “Mean” denotes the mean and “SD” denotes the standard deviation.

TABLE 5
percentage expression of dystroglycan in the keratinocytes of a 50-
year-old donor as a function of the dose of extract used. n = 12; the
Polygonum bistorta extract is obtained according to example 1a) tested at
various dosages as percentage by weight in the final culture medium

| | Mean | SD |
|---|---|---|
| Control | 100 | 22 |
| 0.1% Polygonum bistorta extract | 147 | 3 |
| 0.5% Polygonum bistorta extract | 180 | 6 |
| 1% Polygonum bistorta extract | 245 | 5 |

Conclusions:

The extract according to the invention significantly increased, at the doses tested, the dystroglycan protein expression in the keratinocytes. The extract according to the invention therefore induces an improvement in the structural cohesion of the epithelium, preferentially the epidermis.

### EXAMPLE 5

### Effect of a Polygonum bistorta Extract According to the Invention on the Expression of Dystroglycan in Endothelial Cells

The technique is the same as in example 2, except that the antigen of interest is in this case dystroglycan and the antibody used is an anti-dystroglycan and that it is carried out on endothelial cells and not on keratinocytes.

The results are collated in tables 6 and 7 hereinafter; “Mean” denotes the mean and “SD” denotes the standard deviation.

TABLE 6
percentage expression of dystroglycan in the endothelial cells of a
30-year-old donor as a function of the dose of extract used. n = 12; the
Polygonum bistorta extract is obtained according to example 1a) tested at
various dosages as percentage by weight in the final culture medium

| | Mean | SD |
|---|---|---|
| Control, nontreated | 100 | 41 |
| 0.1% Polygonum bistorta extract | 178 | 13 |
| 0.5% Polygonum bistorta extract | 217 | 12 |
| 1% Polygonum bistorta extract | 390 | 50 |

TABLE 7
percentage expression of dystroglycan in the endothelial cells of a
50-year-old donor as a function of the dose of extract used. n = 12; the
Polygonum bistorta extract is obtained according to example 1a) tested at
various dosages as percentage by weight in the final culture medium

| | Mean | SD |
|---|---|---|
| Control, nontreated | 100 | 22 |
| 0.1% Polygonum bistorta extract | 186 | 19 |
| 0.5% Polygonum bistorta extract | 171 | 12 |
| 1% Polygonum bistorta extract | 187 | 17 |

Conclusions:

The extract according to the invention significantly increased, at the doses tested, the dystroglycan protein expression in the endothelial cells, thereby attesting to its properties in improving the microvascular structural cohesion.

### EXAMPLE 6

### Ex Vivo Evaluation of the Polygonum bistorta Aqueous Extract According to the Invention on the Expression of Perlecan in the Epithelial Basement Membrane, in Particular the Dermoepidermal Junction

The effectiveness of a Polygonum bistorta extract is evaluated on a biopsy of abdominal skin from a 50-year-old woman after immunolabeling of perlecan. The biopsy was placed under survival conditions for a period of 7 days in a specific medium containing the Polygonum bistorta extract obtained according to example 1a) at 0.5% by weight in the culture medium, or not containing said extract (control). The biopsy is emersed, i.e. the epithelium, preferentially the epidermis, is not covered with medium.

The immunofluorescent labelings of perlecan are observed and measured using a confocal microscope by semi-quantitative image analysis.

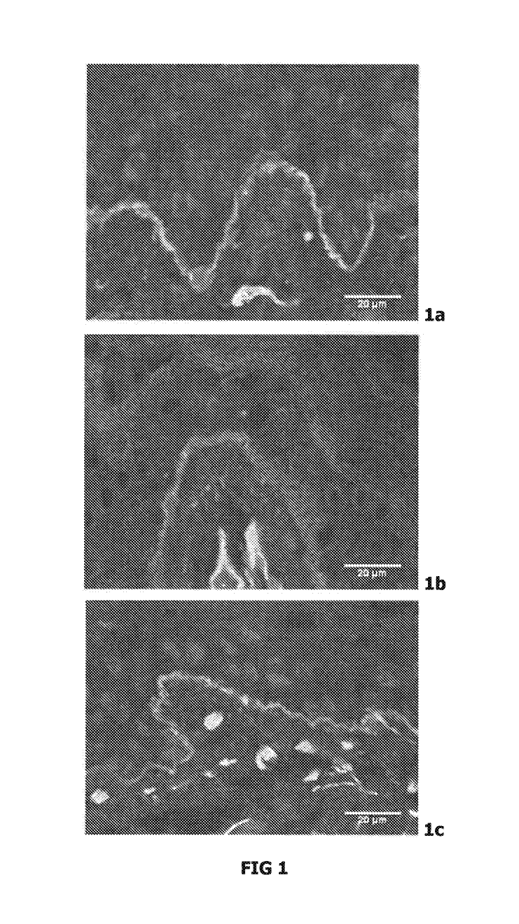

The localization of the fluorescence and the mean intensity of fluorescence reflecting the expression of perlecan at the dermoepidermal junction are studied and measured at the beginning of the experiment and at the end of the experiment. When the dermoepidermal junction is fragmented, a mean of the mean intensities was produced. The results are expressed in arbitrary fluorescence units (AFUs). The measurements were taken as soon as the biopsy was placed in culture, “T0”, and at 7 days of culture, “T7”. The experiment was carried out once (n=1). The results are visualized in FIG. 1. FIG. 1a shows the localization and the intensity of the fluorescence at T0.

FIG. 1b shows the localization and the intensity of the fluorescence in a biopsy at 17 (control).

FIG. 1c shows the localization and the intensity of the fluorescence in the biopsy at 17, i.e. after 7 days of treatment with the extract.

Conclusions:

The fluorescence decreased between T0 (FIG. 1a) and T7 (FIG. 1b) at the dermoepidermal junction, thereby demonstrating that there is a decrease in the expression of perlecan in the dermoepidermal junction over the culture time (control). In the presence of the Polygonum bistorta extract according to the invention, the perlecan protein expression is, on the other hand, maintained over time, as attested to by the fluorescence observed at T7 (FIG. 1c).

### EXAMPLE 7

### Composition Comprising the Extract According to the Present Invention Intended to be Incorporated Into a Cosmetic Composition (Cosmetic Ingredient)

The Polygonum bistorta extract is obtained according to example 1a) and is mixed with the other ingredients of the following formulation:

| | % by weight relative to the total |
|---|---|
| Ingredient | weight of the composition |
| Water | >50% |
| Polygonum bistorta | 0.5-5% |
| aqueous extract (ex 1a) | |
| Hexylene glycol | 1-5% |
| Caprylyl glycol | 0.1-1% |
| Xanthan gum | 0.1-1% |

### EXAMPLE 8

### Clinical Trials of a Composition Comprising an Extract According to the Present Invention Regarding the Radiance of the Skin Complexion and Regarding the Antiwrinkle Effect

The composition of example 7 included in a cream at 1% by weight relative to the total weight of the cosmetic cream was tested on 50 Caucasian subjects, 44 individuals evaluated (half being over 45 years old and half being between 25 and 45 years old) in comparison with 50 other Caucasian subjects using a placebo which consists of a cream that is identical but does not contain the composition of example 7. The cream is applied twice a day to the whole face for 8 weeks. The evaluation of the results was carried out at 4 weeks by imaging, i.e. by image analysis on the basis of macrophotographs taken using the Visia CR® system (Canfield), by a dermatologist who evaluated the effect (scoring), and by means of consumer response to a questionnaire.

Thus, a dermatologist scored the radiance of the complexion and the crows feet wrinkles according to a predefined scale.

The Visia CR® system (Canfield) is an in vivo digital image capture system. Successive analysis of the images obtained made it possible, moreover, to determine several parameters characterizing the effectiveness of the treatment.

The skin texture parameters were evaluated by image analysis. A texture can be described as a distribution of gray levels in the image and more specifically in the regions of interest defined beforehand on the face images. The entropy reflects the complexity of the skin structure. The entropy decreases when the skin becomes more even and therefore equal and softer. The uniformity of the skin was measured by measuring the contrast between the gray levels. Said contrast correlates with the grain of the skin and decreases when the grain of the skin becomes finer.

The two parameters were determined using the Haralick indicators calculated from the co-occurrence matrix.

The evolution of the wrinkles was evaluated on the macrophotographs after spatial alignment of the images between the various times the images were taken. The wrinkles are then identified by means of a projection in the colorimetric space. After thresholding, the wrinkles are identified. A mask corresponding to the regions of interest (ROIs) is defined. The pixels corresponding to the wrinkles are quantified in the same regions of interest in an identical manner before application (T0), after two weeks of treatment (T2 wk) and after four weeks of treatment (T4 wk).

The apparent cumulative length of the crows feet wrinkles was also measured by adding the length of all the wrinkles on a given area. The length is determined via the size of a pixel (1 pixel=0.03 mm). The apparent surface area of the crows feet wrinkles was also measured by calculating the surface area in mm² of the number of segmented pixels (1 pixel=0.03 mm).

The results are presented in tables 8, 9, 10, 11, 12 and 13 hereinafter. “Mean” denotes the mean, “SEM” denotes the standard error of the mean and “VAR” denotes the variation as percentage relative to the control T0. The significance threshold compared with TO is indicated in the “significance” column according to the statistical test mentioned under each table.

TABLE 8
evolution of the radiance of the complexion as mean visual score
given by a dermatologist before application (T0), and after
two weeks (T2wk) and then four weeks (T4wk) of application

| | Mean | SEM | VAR | Significance* |
|---|---|---|---|---|
| Control, nontreated T0 | 1.02 | 0.13 | — | — |
| T2wk | 1.25 | 0.12 | 22% | p < 0.01 |
| T4wk | 1.45 | 0.13 | 42% | p < 0.001 |

*Wilcoxon test

TABLE 9
evolution of the crows feet wrinkles as mean visual score given by a
dermatologist before application (T0), and after two weeks
(T2wk) and then four weeks (T4wk) of application

| | Mean | SEM | VAR | Significance* |
|---|---|---|---|---|
| Control, nontreated T0 | 2.69 | 0.24 | — | — |
| T2wk | 2.36 | 0.21 | −12% | p < 0.001 |
| T4wk | 2.20 | 0.20 | −18% | p < 0.001 |

*Wilcoxon test

TABLE 10
evolution of the softness of the skin (entropy) by image analysis
before application (T0), and after two weeks (T2wk) and then four weeks
(T4wk) of application

| | Mean | SEM | VAR | Significance* |
|---|---|---|---|---|
| Control, nontreated T0 | 5.33 | 0.03 | — | — |
| T2wk | 5.18 | 0.03 | −3% | p < 0.001 |
| T4wk | 5.16 | 0.03 | −3% | p < 0.001 |

*One-way Anova test

TABLE 11
evolution of the uniformity of the skin (contrast) by image analysis
before application (T0), and after two weeks (T2wk), and then four weeks
(T4wk) of application

| | Mean | SEM | VAR | Significance |
|---|---|---|---|---|
| Control, nontreated T0 | 2.16 | 0.11 | — | — |
| T2wk | 1.70 | 0.10 | −21% | p < 0.001 |
| T4wk | 1.67 | 0.10 | −23% | p < 0.001 |

*One-way Anova test

TABLE 12
evolution of the apparent cumulative length of the crows feet
wrinkles in mm by image analysis before application (T0), and after two
weeks (T2wk) and then four weeks (T4wk) of application

| | Mean | SEM | VAR | Significance* |
|---|---|---|---|---|
| Control, nontreated T0 | 566.81 | 34.74 | — | — |
| T2wk | 496.49 | 33.80 | −12% | p < 0.0001 |
| T4wk | 497.00 | 32.26 | −12% | p < 0.0001 |

*One-way Anova test

TABLE 13
evolution of the apparent surface area of the crows feet wrinkles
by image analysis in mm² before application (T0), and after two weeks
(T2wk), and then four weeks (T4wk) of application

| | Mean | SEM | VAR | Significance* |
|---|---|---|---|---|
| Control, nontreated T0 | 138.17 | 8.44 | — | — |
| T2wk | 124.63 | 8.30 | −10% | p < 0.0001 |
| T4wk | 123.50 | 7.97 | −11% | p < 0.0001 |

*One-way Anova test

Conclusions:

The properties of the Polygonum bistorta extract according to the invention were thus observed in vivo and demonstrated. Indeed, it is deduced from table 8 that the extract according to the invention improves the radiance of the complexion after just two weeks of application and that this effect further improves after four weeks of application. This effect is also demonstrated in the results in table 11 which show an improvement in the uniformity of the skin and in the grain of the skin through a decrease in the contrast.

Moreover, the softness of the skin was clearly improved (table 10). Moreover, the extract according to the invention decreases the wrinkles, as is visible in the results in table 9, in particular their apparent length (table 12) and their apparent surface area (table 13), this being significantly.

### EXAMPLE 9

### Demonstration of Claudin-5 by Immunofluorescence on Preconfluent Endothelial Cells

The effectiveness of a Polygonum bistorta extract is evaluated on a monolayer culture of endothelial cells (Lonza) from a biopsy of normal human skin from a 50-year-old woman, and demonstrated after claudin-5 immunofluorescence.

The cells are seeded, cultured in EGM2 medium (Lonza) and amplified for 10 days and then reseeded on an immunohistochemistry plate (Labteck) at 50 000 cells per cm² and placed in the presence of the extract 1a) for 48 h at 0.5% in EGM2 culture medium (Lonza) or brought into contact with the culture medium not containing extract (control).

The immunofluorescent labelings of claudin-5 are observed and measured using a confocal microscope by semi-quantitative image analysis.

The localization of the fluorescence and the mean intensity of fluorescence reflecting the expression of claudin-5 in the endothelial cells are studied and measured at the end of the experiment. The results are expressed as arbitrary fluorescence units (AFUs) and are presented in table 14 hereinafter; “Mean” denotes the mean and “SD” denotes the standard deviation.

TABLE 14

| | Mean | SD |
|---|---|---|
| Control, nontreated | 100 | 2 |
| 0.5% Polygonum bistorta extract | 135 | 4 |

Conclusions:

It was demonstrated, by means of a t-test (p<0.027), that the fluorescence significantly increased in 48 h in the presence of the extract according to the invention. The extract according to the invention therefore makes it possible to increase the claudin-5 protein expression of epithelial cells.

This demonstrates that the extract according to the invention improves the tight junctions at the level of endothelial cells.

### EXAMPLE 10

### Compositions Containing the Polygonum bistorta Extract According to the Invention

Methods known to those skilled in the art are carried out in order to mix together the various parts A, B, C, D, E, or F to prepare a composition according to the present invention. The “products of the invention” represent a Polygonum bistorta extract preferably obtained according to example 1a).

The products of the invention may also be in the form of liposomes containing 5% of soya lecithin and incorporating a quaternized soya solution (600 g final), obtained according to the following embodiment:

30 g of soya lecithin, 12 g of quaternized soya solution and 1.5 g of Polygonum bistorta extract prepared according to example 1a) are placed in a sample tube and diluted in 447 g of pure laboratory water.

After magnetic stirring for 10 minutes at ambient temperature, the mixture is vigorously homogenized for 10 minutes, thus obtaining a liposomal solution in which the liposomes have a mean size that can range between 100 and 800 nanometers depending on the exact homogenization conditions.

The suspension is then left to stir gently for 1 hour. 90 g of butylene glycol, 6 g of phenoxyethanol and 6 g of hydroxyethylcellulose (gelling agent) are then added.

Cosmetic formulation 10a:

| A | Water | qs 100 |
|---|---|---|
| | Butylene Glycol | 2 |
| | Glycerin | 3 |
| | Sodium Dihydroxycetyl | 2 |
| | Phosphate, | |
| | Isopropyl Hydroxycetyl Ether | |
| B | Glycol Stearate SE | 14 |
| | Triisononanoin | 5 |
| | Octyl Cocoate | 6 |
| C | Butylene Glycol, | 2 |
| | Methylparaben, | |
| | Ethylparaben, Propylparaben, | |
| | pH adjusted to 5.5 | |
| D | Products of the invention | 0.01-10% |

Cosmetic formulation 10b:

| A | Water | qs 100 |
|---|---|---|
| | Butylene Glycol | 2 |
| | Glycerin | 3 |
| | Polyacrylamide, Isoparaffin, | 2.8 |
| | Laureth-7 | |
| B | Butylene Glycol, | 2 |
| | Methylparaben, | |
| | Ethylparaben, Propylparaben; | |
| | Phenoxyethanol, | 2 |
| | Methylparaben, | |
| | Propylparaben, Butylparaben, | |
| | Ethylparaben | |
| | Butylene Glycol | 0.5 |
| D | Products of the invention | 0.01-10% |

Cosmetic formulation 10c:

| | | |
|---|---|---|
| A | Carbomer | 0.50 |
| | Propylene Glycol | 3 |
| | Glycerol | 5 |
| | Water | qs 100 |
| B | Octyl Cocoate | 5 |
| | Bisabolol | 0.30 |
| | Dimethicone | 0.30 |
| C | Sodium Hydroxide | 1.60 |
| D | Phenoxyethanol, | 0.50 |
| | Methylparaben, | |
| | Propylparaben, Butylparaben, | |
| | Ethylparaben | |
| E | Fragrance | 0.30 |
| F | Products of the invention | 0.01-10% |

Dermatological Formulation 10D in the Form of an Ointment

| A | Excipients | |
|---|---|---|
| | Low-density polyethylene | 5.5 |
| | Liquid paraffin | qs 100 |
| B | Product of the invention* | 0.001-0.1 |

*The Polygonum bistorta extract is the one described in example 1a) followed by a sterilization and drying step.

1.-21. (canceled)

22. A method of cosmetic care for stimulating the expression of perlecan and/or dystroglycan, comprising the topical administration of an effective amount of a Polygonum bistorta extract, to at least one concerned area of healthy skin and/or healthy mucous membrane and/or healthy scalp in need thereof.

23. The method as claimed in claim 22, wherein the Polygonum bistorta extract is obtained by aqueous extraction.

24. The method as claimed in claim 22, wherein the Polygonum bistorta extract is obtained by extraction from the Polygonum bistorta root.

25. The method as claimed in claim 22, for stimulating perlecan and/or dystroglycan protein expression.

26. The method as claimed in claim 22, for preventing and/or combating ageing of the skin and/or of the mucous membranes and/or of the scalp, for preventing and/or combating a decrease in homeostasis of the skin and/or of the mucous membranes and/or of the scalp and/or for improving it, for reinforcing the epithelial basement membrane of the skin and/or of the mucous membranes and/or of the scalp, for improving keratinocyte proliferation and/or differentiation, for preventing and/or combating a decrease in vascularization of the skin and/or of the mucous membranes and/or of the scalp and/or for improving it, for improving the morphogenesis of the epithelium, of the skin and/or of the mucous membranes and/or of the scalp, for restoring the epithelial architecture of the skin and/or of the mucous membranes and/or of the scalp, for improving the complexion of the skin and/or of the mucous membranes, for improving the firmness and/or the density of the skin and/or of the mucous membranes and/or of the scalp, and/or for combating a decrease in the thickness of the epithelium of the skin and/or of the mucous membranes and/or of the scalp, and/or for increasing the thickness of the epithelium of the skin and/or of the mucous membranes and/or of the scalp.

27. The method as claimed in claim 26, for improving the skin complexion by eliminating red patches and/or by making the complexion uniform and/or by giving it a luminous, radiant, healthy and/or nourished appearance, a well-looking effect and/or a pinkish radiance.

28. The method as claimed in claim 26, for preventing and/or combating skin ageing, by decreasing or suppressing wrinkles and/or fine lines.

29. The method as claimed in claim 26, for preventing and/or combating a decrease in the homeostasis of the skin and/or of the mucous membranes and/or of the scalp and/or for improving the homeostasis of the skin and/or of the mucous membranes and/or of the scalp.

30. The method as claimed in claim 26, for increasing the thickness of the epithelium of the skin and/or of the mucous membranes and/or of the scalp.

31. The method as claimed in claim 26, for improving the firmness and/or the density of the skin and/or of the mucous membranes and/or of the scalp.

32. The method as claimed in claim 22, wherein the concerned area of the skin is chosen from the face, the neck, the neckline, the bust and/or the hands.

33. The method as claimed in claim 32, wherein the Polygonum bistorta extract is present in a cosmetic composition comprising a cosmetically acceptable excipient.

34. The method as claimed in claim 22, wherein the Polygonum bistorta extract is solubilized in an aqueous solution comprising hexylene glycol, caprylyl glycol or a mixture thereof.

35. The method as claimed in claim 34, wherein the Polygonum bistorta extract is solubilized in a content of between 0.1% and 10% by weight relative to the total weight of the aqueous solution.

36. The method as claimed in claim 22, for stimulating the expression of perlecan and/or dystroglycan in keratinocytes and/or endothelial cells of the skin and/or of the mucous membranes and/or of the scalp.

37. The method as claimed in claim 22, wherein the cosmetic composition is an aqueous or oily solution, a cream or an aqueous gel or an oily gel; a milk; an emulsion; a microemulsion or a nanoemulsion; a mask; a serum; a lotion; a liquid soap; a dermatological bar; an ointment; a foam; a patch or an anhydrous product.

38. A method of treatment and/or prevention of rosacea, telangiectasias, chapping, and/or pathological conditions of the buccal and/or ocular mucous membranes comprising the topical administration of an effective amount of a Polygonum bistorta extract or a dermatological composition containing a Polygonum bistorta extract and a dermatologically acceptable excipient to a patient in need thereof.

39. The method as claimed in claim 38, wherein the Polygonum bistorta extract is obtained by aqueous extraction or by extraction from the Polygonum bistorta root.

40. The method as claimed in claim 33, wherein the Polygonum bistorta extract is present in a content of between 1×10⁻⁴ and 10% by weight relative to the total weight of the composition.

41. The method as claimed in claim 26 for improving keratinocyte proliferation and/or differentiation associated with ageing of the skin and/or the mucous membranes and/or the scalp and/or for restoring the epithelial architecture of the skin and/or the mucous membranes and/or the scalp having undergone ageing.
